(19) United States
(12) Patent Application Publication
(10) Pub. No.: US 2016/0245790 A1
(43) Pub. Date: Aug. 25, 2016

(54) DEVICE FOR THERMALLY DENATURING BIOMOLECULE AND METHOD FOR PRODUCING DEVICE

(71) Applicants: Osaka University, Osaka Prefecture (JP); Quantum Biosystems Inc., Tokyo (JP)

(72) Inventors: Tomoji Kawai, Osaka Prefecture (JP); Masayuki Furuhashi, Osaka Prefecture (JP); Masateru Kawaguchi, Osaka Prefecture (JP); Mark Oldham, Emerald Hills, CA (US); Eric Nordman, Palo Alto, CA (US)

(21) Appl. No.: 15/048,889

(22) Filed: Feb. 19, 2016

Related U.S. Application Data
(63) Continuation of application No. PCT/IB2014/002128, filed on Aug. 26, 2014.

(30) Foreign Application Priority Data
Aug. 27, 2013 (JP) 2013-175637

(51) Int. Cl. G01N 33/487; G01N 27/26; C12Q 1/68; B01L 3/00; B01L 7/00

(52) U.S. Cl. CPC G01N 33/48721; B01L 3/502715; B01L 7/52; C12Q 1/6869; G01N 27/26; B01L 2300/0896; B01L 2300/1827; B01L 2300/04; B01L 2300/12; B01L 2300/0645

(57) ABSTRACT

The present disclosure provides methods and systems that can reduce the amount of sample necessary to detect or identify, or both detect and identify, a biomolecule, and increase the rate of denaturing of the biomolecule. A device for thermally denaturing a biomolecule may include: a substrate having low thermal conductivity; a heater disposed adjacent to the substrate; a temperature sensor disposed adjacent to the substrate; a semiconductor oxide film disposed adjacent to the substrate, a nanochannel formed in a region of the semiconductor oxide film, and a cover over the nanochannel.

### CROSS-REFERENCE

This application is a Continuation Application of International Patent Application No. PCT/IB2014/002128, which claims priority to Japanese Patent Application No. JP 2013-175637, filed Aug. 27, 2013, which is entirely incorporated herein by reference.

### BACKGROUND

Nanopores (or nano-gaps) may be useful for detecting a biomolecule, including determining the sequence of a nucleic acid molecule, such as a deoxyribonucleic acid (DNA) or ribonucleic acid (RNA) molecule. The determination of the sequence of a nucleic acid molecule may provide various benefits, such as aiding in diagnosing and/or treating a subject. For example, the nucleic acid sequence of a subject may be used to identify, diagnose and potentially develop treatments for genetic diseases.

### SUMMARY

A device for detecting or identifying a biomolecule by the use of a microchannel has widely contributed to increases in the rate of analysis and to reductions in amount of sample necessary. In analysis of DNA, which is an example of a biomolecule, the DNA may be treated by heating to a high temperature and be processed into single strands for procedures such as amplification (e.g., polymerase chain reaction (PCR)) and hybridization. In the same way, when analyzing a protein, which is another example of a biomolecule, the protein may be processed into short peptide fragments.

However, in the conventional example described above, since the depth of the channel is determined by the thickness of the silicon substrate, it may not be possible to make the channel shallower. Thus, the volume of a chamber heated by a heater is large and denaturation of a biomolecule takes a substantial amount of time.

The present disclosure provides devices, systems and methods for thermally denaturing a biomolecule (e.g., DNA or RNA). Devices, systems and methods of the present disclosure reduce the amount of sample needed to detect or identify, or both detect and identify, a biomolecule and to increase the rate of denaturing of the biomolecule.

In some embodiments wherein a device may be utilized for thermally denaturing a biomolecule the device may include: a substrate having low thermal conductivity; a resistive heater disposed on the substrate; a temperature sensor disposed in juxtaposition with the heater on the substrate; a silicon oxide film layered on substrate, heater, and temperature sensor; a covering member overlapped on silicon oxide film; and a nanochannel, formed in a region in silicon oxide film, the region overlapping with a heater, and the region also overlapping with a temperature sensor.

In some embodiments, wherein a device may be utilized for thermally denaturing a biomolecule, a heater is a resistive heater, and the temperature thereof may be raised by Joule heating when a voltage is applied and current passes therethrough. The amount of Joule heating may be proportional to the square of an applied voltage. Since the size of the heater may be reduced such that a heater may overlap with a nanochannel, power density of Joule heating can be increased. Thus, the temperature of a nanochannel disposed on a heater can be raised using less Joule heating, and heat conduction into surroundings may be reduced. Since a heater may be disposed on a substrate having low thermal conductivity, heat conduction through a substrate may also be suppressed.

In addition, since a small heater has a low heat capacity, it may reach a constant temperature in a short time. Thus, rapid temperature modulation (heating) may be possible. Temperature regulation may be performed by the use of a temperature sensor. Since heater and temperature sensor may be covered by a silicon oxide film and a nanochannel may be formed in a silicon oxide film, a biomolecule passing through a nanochannel can be locally heated rapidly and be consistently denatured. Since a nanochannel may be a channel having a depth equal to or less than 1 μm, the amount of sample necessary to detect or identify, or both detect and identify, a biomolecule may be reduced.

In some embodiments wherein a device may be utilized for thermally denaturing a biomolecule, a plurality of columnar parts may be provided in a nanochannel, wherein at least two columnar parts may be aligned in a longitudinal direction and wherein at least two columnar parts may be aligned in a width direction of a nanochannel.

In some embodiments wherein a device may be utilized for thermally denaturing a biomolecule, an entangled biomolecule may be linearized while passing among multiple columnar parts aligned in a nanochannel. A biomolecule may be heated by a heater and be denatured, as described above, enabling a rate of detecting or identifying, or both detecting and identifying, of a biomolecule at a molecular level to be increased.

In some embodiments where a device for thermally denaturing a biomolecule may be utilized, a heater and a temperature sensor may be arranged in a width direction of a nanochannel.

In some embodiments, a device for thermally denaturing a biomolecule as described herein, parameters for temperature regulation using a temperature sensor can be minimized. Thus, complex temperature regulation may not be necessary.

In some embodiments wherein a device for thermally denaturing a biomolecule may be utilized, for which a heater and a temperature sensor may be arranged in a longitudinal direction of a nanochannel.

In some embodiments, wherein a device may be utilized for thermally denaturing a biomolecule, a heater may be fully disposed in a width direction of a nanochannel. Thus, a biomolecule flowing through a nanochannel may be efficiently heated and denatured.

In some embodiments, a method for producing a device for thermally denaturing a biomolecule may include: disposing a resistive heater on a substrate having low thermal conductivity; disposing a temperature sensor in juxtaposition with a heater on a substrate; layering a silicon oxide film on a substrate, a heater, and a temperature sensor; forming a nanochannel in a region of a silicon oxide film, the region overlapping with a heater, the region also overlapping with a temperature sensor; and overlapping a covering member on a silicon oxide film.

In some embodiments, wherein a method for producing a device for denaturing a biomolecule may be utilized, it may be possible to produce a device for denaturing a biomolecule that requires less sample for detection or identification, or both, of a biomolecule than may be required without the utilization of the method for denaturation, and that can increase the rate of denaturing of a biomolecule.

In some embodiments, a method for producing a device for thermally denaturing a biomolecule may be utilized wherein the method may include: preparing a plurality of columnar parts in a nanochannel, which may have at least two columnar parts aligned in a longitudinal direction and may have at least two columnar parts aligned in a width direction of a nanochannel.

In some embodiments wherein a device may be utilized for thermally denaturing a biomolecule, it may be possible to produce a device for thermally denaturing a biomolecule that can increase the rate of detection or identification, or both, of a biomolecule at a molecular level.

As described above, according in some embodiments, it is possible to obtain advantageous effects by which less sample may be needed for detection or identification, or for both detection and identification, of a biomolecule, and in that the rate of denaturing of a biomolecule may be increased.

An aspect of the present disclosure provides a device for thermally denaturing a biomolecule, comprising a substrate having low thermal conductivity; a resistive heater disposed adjacent to the substrate; a temperature sensor disposed in juxtaposition with the resistive heater adjacent to the substrate; a semiconductor oxide film adjacent to the resistive heater and the temperature sensor; a nanochannel formed in at least a portion of the semiconductor oxide film; and a covering member over at least a portion of the nanochannel. In an embodiment, the nanochannel overlaps the resistive heater and the temperature sensor. In some cases, the nanochannel may be sealed with a cover, such as, for example, hermetically sealed.

In an embodiment, the device further comprises one or more columnar parts in the nanochannel. In another embodiment, the one or more columnar parts comprise a plurality of columnar parts. In another embodiment, at least two columnar parts of the plurality are aligned along a longitudinal direction of the nanochannel, and wherein at least two columnar parts of the plurality are aligned along a width direction of the nanochannel. The resistive heater and/or temperature sensor may be before, adjacent to, or after the one or more columnar parts (e.g., along the length of the nanochannel).

In an embodiment, the resistive heater and the temperature sensor are arranged along a width direction of the nanochannel. In another embodiment, the resistive heater and the temperature sensor are arranged along a longitudinal direction of the nanochannel. In another embodiment the resistive heater and the temperature sensor are interdigitated.

In an embodiment, the device further comprises at least one pair of electrodes in fluid communication with the nanochannel, wherein the pair of electrodes detects a current across the nanochannel. In another embodiment, the current is tunneling current. In another embodiment, the at least one pair of electrodes is in the nanochannel. In another embodiment, the at least one pair of electrodes is separated by a gap having a distance that is less than or equal to about 2 nanometers. In another embodiment, the distance is less than or equal to about 1 nanometer. In another embodiment, the distance is greater than about 0.5 nanometers. In another embodiment, the at least one pair of electrodes is separated by a gap having a distance that is less than a diameter of the biomolecule.

In an embodiment, the biomolecule is a nucleic acid molecule. In another embodiment, the nucleic acid molecule is deoxyribonucleic acid, ribonucleic acid, or a variant thereof. In another embodiment, the biomolecule is suspended in a low ionic concentration fluid. The low ionic concentration fluid may be in the nanochannel. The low ionic concentration fluid may increase the persistence length.

In an embodiment, the resistive heater is proximate to the nanochannel. In another embodiment, the resistive heater overlaps the nanochannel. In another embodiment, the resistive heater is adapted for use in heating and temperature sensing. In another embodiment, the device further comprises a plurality of resistive heaters that generate at least two temperature zones. In another embodiment, the temperature zones are different temperature zones. For instance, the temperature zones have different temperatures or temperature ranges.

In an embodiment, the semiconductor oxide film comprises silicon oxide.

In an embodiment, the substrate has a thermal conductivity that is less than or equal to about 100 W/(mK). In another embodiment, the substrate has a thermal conductivity that is less than or equal to about 10 W/(mK). In another embodiment, the substrate has a thermal conductivity that is less than or equal to about 5 W/(mK).

Another aspect of the present disclosure provides a method, comprising (a) providing a device having (i) a substrate having low thermal conductivity, (ii) a resistive heater disposed adjacent to the substrate, (iii) a temperature sensor disposed in juxtaposition with the resistive heater adjacent to the substrate, (iv) a semiconductor oxide film adjacent to the resistive heater and the temperature sensor, (v) a nanochannel formed in at least a portion of the semiconductor oxide film, and (vi) a covering member over at least a portion of the nanochannel; (b) directing the biomolecule through the nanochannel; and (c) using the resistive heater to apply heat to the biomolecule. In an embodiment, the nanochannel overlaps the resistive heater and the temperature sensor. In some cases, the nanochannel may be sealed with a cover, such as, for example, hermetically sealed.

In an embodiment, the device further comprises one or more columnar parts in the nanochannel. In another embodiment, the one or more columnar parts comprise a plurality of columnar parts. In another embodiment, at least two columnar parts of the plurality are aligned along a longitudinal direction of the nanochannel, and wherein at least two columnar parts of the plurality are aligned along a width direction of the nanochannel. The resistive heater and/or temperature sensor may be before, adjacent to, or after the one or more columnar parts (e.g., along the length of the nanochannel).

In an embodiment, the resistive heater and the temperature sensor are arranged along a width direction of the nanochannel. In another embodiment, the resistive heater and the temperature sensor are arranged along a longitudinal direction of the nanochannel. In another embodiment the resistive heater and the temperature sensor are interdigitated.

In an embodiment, the device further comprises at least one pair of electrodes in fluid communication with the nanochannel. The at least one pair of electrodes may be adapted to detect a current across the nanochannel. The current may be tunneling current. In another embodiment, the at least one pair of electrodes is in the nanochannel. In another embodiment, the at least one pair of electrodes is separated by a gap having a distance that is less than or equal to about 2 nanometers. In another embodiment, the distance is less than or equal to about 1 nanometer. In another embodiment, the distance is greater than about 0.5 nanometers. In another embodiment, the at least one pair of electrodes is separated by a gap having a distance that is less than a diameter of the biomolecule. In another embodiment, the method further comprises using the at least one pair of electrodes to measure a current across a gap that separates the at least one pair of electrodes. In another embodiment, the current is a tunneling current. The tunneling current may be across the biomolecule. Such tunneling may be quantum mechanical tunneling.

In an embodiment, the biomolecule is a nucleic acid molecule. In another embodiment, the nucleic acid molecule is deoxyribonucleic acid, ribonucleic acid, or a variant thereof. In another embodiment, the biomolecule is suspended in a low ionic concentration fluid. The low ionic concentration fluid may increase the persistence length.

In an embodiment, the resistive heater is proximate to the nanochannel. In another embodiment, the resistive heater overlaps the nanochannel. In another embodiment, the resistive heater is adapted for use in heating and temperature sensing.

In an embodiment, the device further comprises a plurality of resistive heaters that generate at least two temperature zones. In another embodiment, the temperature zones are different temperature zones. For instance, the temperature zones have different temperatures or temperature ranges.

In an embodiment, the semiconductor oxide film comprises silicon oxide.

In an embodiment, the substrate has a thermal conductivity that is less than or equal to about 100 W/(mK). In another embodiment, the substrate has a thermal conductivity that is less than or equal to about 10 W/(mK). In another embodiment, the substrate has a thermal conductivity that is less than or equal to about 5 W/(mK).

Another aspect of the present disclosure provides a method for forming a device that thermally denatures a biomolecule, comprising (a) disposing a resistive heater adjacent to a substrate having low thermal conductivity; (b) disposing a temperature sensor in juxtaposition with the resistive heater adjacent to the substrate; (c) providing a semiconductor oxide film adjacent to the substrate, the resistive heater and the temperature sensor; (d) forming a nanochannel in at least a portion of the semiconductor oxide film; and (e) providing a covering member over at least a portion of the nanochannel. In an embodiment, disposing a resistive heater adjacent to a substrate having low thermal conductivity comprises depositing the resistive heater. In another embodiment, disposing a temperature sensor in juxtaposition with the resistive heater adjacent to the substrate comprises forming the temperature sensor. In some cases, the nanochannel may be sealed with the covering member, such as, for example, hermetically sealed.

In an embodiment, the nanochannel overlaps the resistive heater and the temperature sensor.

In an embodiment, the method further comprises forming one or more columnar parts in the nanochannel. In another embodiment, forming the one or more columnar parts comprises forming a plurality of columnar parts. In another embodiment, at least two columnar parts of the plurality are aligned along a longitudinal direction of the nanochannel, and wherein at least two columnar parts of the plurality are aligned along a width direction of the nanochannel. The resistive heater and/or temperature sensor may be before, adjacent to, or after the one or more columnar parts (e.g., along the length of the nanochannel).

In an embodiment, the resistive heater and the temperature sensor are arranged along a width direction of the nanochannel. In another embodiment, the resistive heater and the temperature sensor are arranged along a longitudinal direction of the nanochannel. In another embodiment the resistive heater and the temperature sensor are interdigitated.

In an embodiment, the method further comprises forming at least one pair of electrodes in fluid communication with the nanochannel, wherein the pair of electrodes detects a current across the nanochannel. In another embodiment, the at least one pair of electrodes is in the nanochannel. In another embodiment, the at least one pair of electrodes is separated by a gap having a distance that is less than or equal to about 2 nanometers. In another embodiment, the distance is less than or equal to about 1 nanometer. In another embodiment, the distance is greater than about 0.5 nanometers. In another embodiment, the at least one pair of electrodes is separated by a gap having a distance that is less than a diameter of the biomolecule.

In an embodiment, the biomolecule is a nucleic acid molecule. In another embodiment, the nucleic acid molecule is deoxyribonucleic acid, ribonucleic acid, or a variant thereof.

In an embodiment, the resistive heater is proximate to the nanochannel. In another embodiment, the resistive heater overlaps the nanochannel. In another embodiment, the resistive heater is adapted for use in heating and temperature sensing.

In an embodiment, the method further comprises forming a plurality of resistive heaters that generate at least two temperature zones. In another embodiment, the temperature zones have different temperatures or temperature ranges.

In an embodiment, the semiconductor oxide film comprises silicon oxide.

In an embodiment, the substrate has a thermal conductivity that is less than or equal to about 100 W/(mK). In another embodiment, the substrate has a thermal conductivity that is less than or equal to about 10 W/(mK). In another embodiment, the substrate has a thermal conductivity that is less than or equal to about 5 W/(mK).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 is a plan view showing a device for thermally denaturing a biomolecule, in which a heater and a temperature sensor are arranged along a width direction of a channel;

FIG. 2 is a cross sectional view of FIG. 1 viewed in section 2-2 (along the width), which shows a device for thermally denaturing a biomolecule;

FIG. 3 is a plan view showing a device for thermally denaturing a biomolecule, in which a heater and a temperature sensor are arranged in a longitudinal direction of a channel;

FIG. 4 is a diagram showing experimental results; and

FIGS. 5A-5C show different arrangements for associated heaters and temperature sensors.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “gap,” as used herein, generally refers to a pore, channel or passage formed or otherwise provided in a material. The material may be a solid state material, such as a substrate. The gap may be disposed adjacent or in proximity to a sensing circuit or an electrode coupled to a sensing circuit. In some examples, a gap has a characteristic width or diameter on the order of 0.1 nanometers (nm) to about 1000 nm. A gap having a width on the order of nanometers may be referred to as a “nano-gap.”

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be part of a nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded. A nucleic acid may be native or modified. A modified nucleic acid may include natural modifications, such as methylation, as well as manmade (or unnatural) modifications.

The term “silicon oxide” or “oxide,” as used herein, generally refers to electrical insulators such as silicon monoxide, silicon dioxide, silicon nitride, and oxides of other metals or semiconductors. In some examples, a silicon oxide is SiOx, where ‘x’ is a number greater than zero.

The term “substrate,” as used herein, generally refers to a material on or adjacent to which a device, such as a heater, is deposited. A substrate may comprise silicon wafers with insulating layers such as silicon dioxide, silicon nitride, plastics or other low conductivity materials.

The term “resistive heater,” as used herein, generally refers to a conductor that releases heat upon the passage (or flow) of an electric current through the conductor. Such heating may be referred to as Joule heating, ohmic heating or resistive heating. The amount of heat released may be proportional to the square of the current times the resistance of the resistive heater. A resistive heater can include a heating element that is configured to release heat upon the flow of electrical current therethrough. Examples of heating elements include nichrome 80/20 (80% nickel, 20% chromium), kanthal (FeCrAl alloy), and cupronickel (CuNi alloy). A heating element may be a wire, ribbon or strip. A heating element may be coiled or flat.

The term “temperature sensor,” as used herein, generally refers to any sensor capable of measuring temperature. An example of a temperature sensor is a resistive thermal device (RTD), thermistor, or thermocouple. In some examples, a thermocouple can include a nickel alloy, platinum/rhodium alloy, tungsten/rhenium, chromel-gold/iron alloy, noble metal alloy, platinum/molybdenum alloy, or iridium/rhodium alloy. In an example, a thermocouple is a chromel-alumel thermocouple. Chromel is an alloy comprising about 90 percent nickel and 10 percent chromium. Alumel is an alloy comprising about 95% nickel, 2% manganese, 2% aluminium and 1% silicon. As an alternative, a temperature sensor may be optical, such as a detector of infrared (IR) radiation.

The term “nanochannel,” as used herein, generally refers to an open or closed channel with a width less than or equal to about 1000 nanometers (nm). A nanochannel can be a structure that direct fluid flow from one point to another, such as across electrodes for measuring tunneling current across a gap.

The term “nanoelectrode,” as used herein, generally refers to an electrode that is adapted to detect a current, such as tunneling current. The term “nanoelectrode pair,” as used herein, generally refers to a pair of electrodes spaced apart wherein the separation is less than about 1000 nm, 100 nm, 10 nm, 2 nm, 1 nm, 0.9 nm, 0.8 nm, 0.7 nm, 0.6 nm or 0.5 nm.

The term “linearization feature,” as used herein, generally refers to features utilized to untangle a nucleic acid molecule (e.g., DNA) and send a resultant linearized nucleic acid molecule down a channel in a linear fashion or configuration. Linearization features may include columnar features, channel width or depth variations, or other features to generate linear nucleic acid fragments.

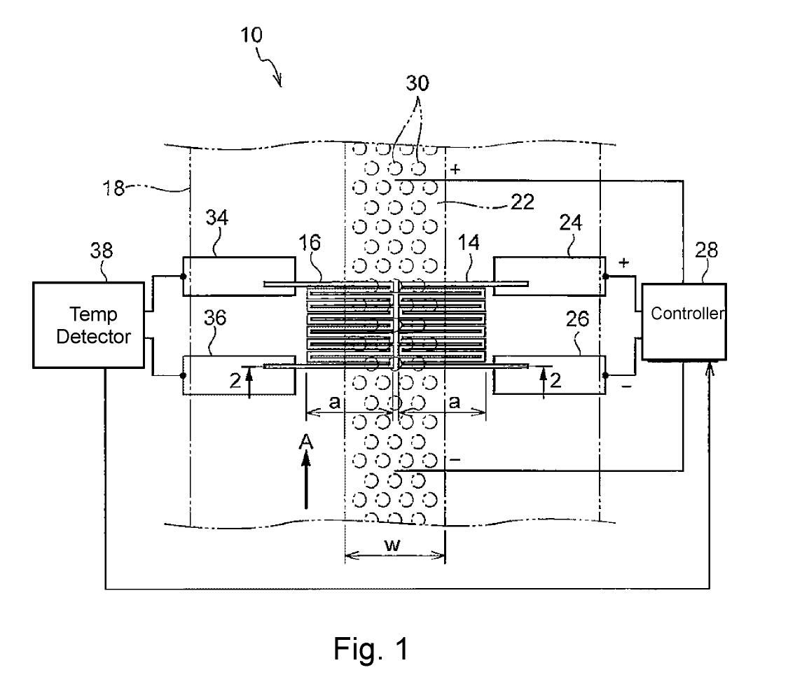

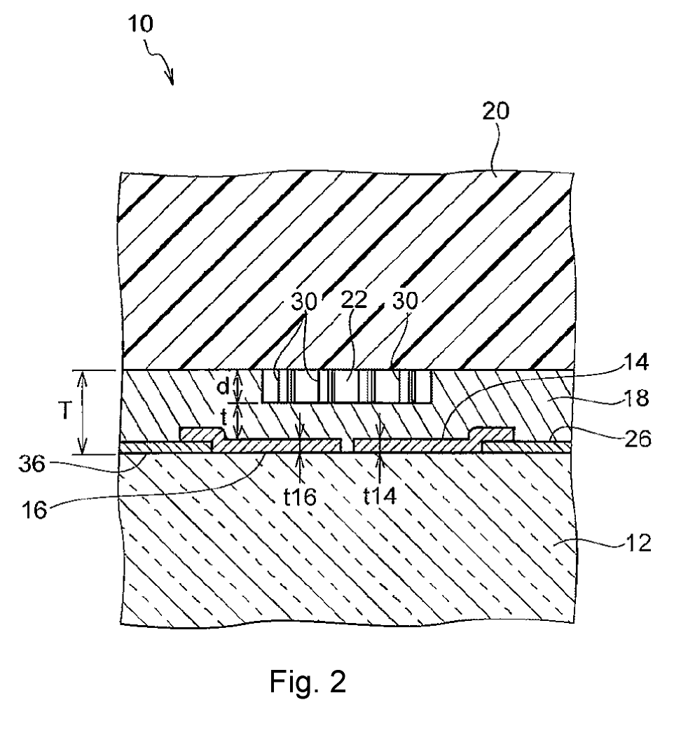

In some embodiments as shown variously in FIG. 1 and FIG. 2, a device 10 for thermally denaturing a biomolecule may include a substrate 12 having a low thermal conductivity, a heater 14, a temperature sensor 16, a silicon oxide film 18, a covering member 20, and a nanochannel 22. A biomolecule may be, for example, a DNA or a peptide.

A material (e.g., substrate 12) with low thermal conductivity may have a thermal conductivity that is less than or equal to about 500 W/(mK), 400 W/(mK), 300 W/(mK), 200 W/(mK), 100 W/(mK), 50 W/(mK), 40 W/(mK), 30 W/(mK), 20 W/(mK), 10 W/(mK), 9 W/(mK), 8 W/(mK), 7 W/(mK), 6 W/(mK), 5 about W/(mK), 4 W/(mK), 3 W/(mK), 2 W/(mK) or 1 W/(mK). In some examples, a material with low thermal conductivity has a thermal conductivity that is less than that of silicon. In some examples, a low thermal conductivity material has a thermal conductivity from about 0.1 W/(mK) to 200 W/(mK), 0.1 W/(mK) to 100 W/(mK), or 0.1 W/(mK) to 10 W/(mK). Such thermal conductivities may be as measured at 25° C. In some situations, the material of the substrate 12 comprises glass, quartz, polypropylene, or the like.

In some embodiments as shown in FIG. 2, a heater 14 may be disposed on a substrate 12, and may be a resistive heater. A heater 14 may be, for example, a microheater made of platinum. On a substrate 12, electrodes 24, 26, which may be respectively connected to an end of a heater 14, may be provided. Electrodes 24, 26 may be connected to a controller 28. Voltage and or current controlled by controller 28 may be applied to electrodes 24, 26.

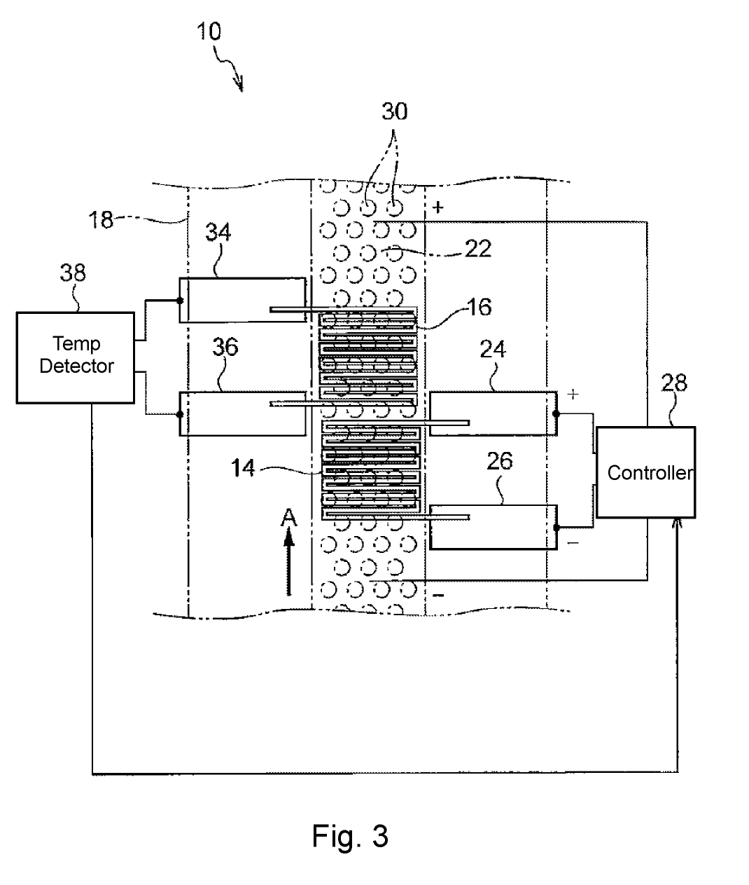

In some embodiments as shown in FIG. 2, temperature sensor 16 may be disposed in juxtaposition with heater 14 on substrate 12. Temperature sensor 16 may be, for example, a resistive temperature sensor made of platinum. On substrate 12, electrodes 34, 36, which may be respectively connected to an end of temperature sensor 16, may be provided. In some embodiments as shown in FIG. 1, heater 14 and temperature sensor 16 may be arranged in a width direction of nanochannel 22. In some embodiments, as shown in FIG. 3, heater 14 and temperature sensor 16 may be arranged in a longitudinal direction (or axis) of nanochannel 22. Thermometer 16 may be connected to a temperature detector 38. A measurement by temperature detector 38 may be fed back to controller 28 for use in controlling a temperature.

In FIG. 1, temperature sensor 16 and heater 14 may be formed in nearly square shape when viewed in plan view. A length “a” of a side of temperature sensor 16 and heater 14 may, for example, be from 5 to 100 μm. In some embodiments as shown in FIG. 2, thicknesses t14 and t16 may be, for example, from 10 to 100 nm.

As shown in FIG. 2, silicon oxide film 18 may be a silicon dioxide thin film, which may be layered on substrate 12, heater 14, and temperature sensor 16. A thickness “T” of silicon oxide film 18 with respect to a surface of substrate 12 may be deeper than a depth “d” of nanochannel 22 and may be, for example, from 0.1 μm to 2 μm.

Covering member 20 may be at least partially or wholly overlap the silicon oxide film 18. Covering member 20 may comprise glass, SU8, polydimethylsiloxane (PDMS), or the like. Covering member 20 may be a covering part of nanochannel 22.

Nanochannel 22 may be formed in a region of silicon oxide film 18, the region may overlap heater 14, and the region may also overlap temperature sensor 16. Nanochannel 22 may be a groove having a depth equal to or less than 1 μm (i.e., on the order of nanometers). In particular, the depth of nanochannel 22 may be, for example, from 10 nm to 1000 nm. In some embodiments as shown in FIG. 1, a width “w” of a nanochannel 22 may be, for example, from 0.5 μm to 100 μm. In other embodiments as shown in FIG. 2, silicon oxide film 18 may be interposed between heater 14 and nanochannel 22 and between temperature sensor 16 and nanochannel 22. Nanochannel 22 may be provided with an inlet and an outlet (not shown) for a solution containing biomolecules. A solution may flow in the direction of arrow “A,” for example, at least in part by electrophoresis, under the control of controller 28.

A plurality of columnar parts 30 may be provided in nanochannel 22, wherein at least two columnar parts 30 may be aligned in a longitudinal direction and wherein at least two columnar parts 30 may be aligned in a width direction of nanochannel 22. A height of columnar part 30 may be equal to a depth “d” of nanochannel 22, or may be of height less than a depth d of nanochannel 22. Since columnar part 30 may be a pillar (a column) on the order of nanometers, it may be called a “nanopillar.” Columnar part 30 may have, for example, a circular cylindrical shape, a hexagonal shape or other shapes, and a diameter thereof may be freely chosen. A diameter of a columnar part 30 may be reduced further so that a number of columnar parts 30 can be increased. A set of nanopillars may be considered to be a linearization feature. Different members of the linearization feature may have similar sizes and shapes, or may have different sizes and shapes, and may be spaced with a regular spacing, a linearly or otherwise regularly changing spacing, or with irregular spacing or irregularly changing spacing.

Although certain components of devices herein have been described as include silicon oxide, it will be appreciated that other materials maybe used. Such other materials may be thermal and/or electrical insulators, and may include, for example, other semiconductor or metal oxides.

### Operation

Devices and systems of the present disclosure may be used in various applications. In some cases, devices and systems of the present disclosure may be used to thermally denature a biomolecule, such as a nucleic acid molecule. In some examples, the device 10 of FIG. 1 may be utilized for thermally denaturing. In such a case, the heater 14 may be a resistive heater, and the temperature thereof may be raised by Joule heating when voltage is applied and current passes therethrough. The amount of Joule heating may be proportional to the square of the applied voltage. Since the size of heater 14 may be reduced so that heater 14 may overlap nanochannel 22, power density of Joule heating may be increased. Thus, the temperature of nanochannel 22 disposed on heater 14 can be raised using less Joule heating, and heat conduction into the surroundings can be reduced. Since heater 14 may be disposed on a substrate 12 having low thermal conductivity, heat conduction through substrate 12 may also be suppressed. Therefore, localized heating by heater 14 may be possible.

In addition, since heater 14 may be small, and may have a low heat capacity, heater 14 may be utilized to reach a constant temperature in a short time. Thus, rapid temperature modulation (heating) may be possible. Temperature regulation may be performed utilizing temperature sensor 16 so that a solution of biomolecules reaches, for example, 95° C., or another temperature associated with denaturation of a DNA sample in a given solution, which may be a low ionic concentration solution. Since heater 14 and temperature sensor 16 may be covered by silicon oxide film 18 and nanochannel 22 may be formed in silicon oxide film 18, a biomolecule passing through nanochannel 22 may be locally heated rapidly and be readily denatured. Since nanochannel 22 may be a channel having a depth equal to or less than 1 μm, less sample may be needed to detect or identify, or both detect and identify, a biomolecule.

A sample of biomolecule(s) is typically in an entangled state to some extent. Biomolecules may be disentangled when passing through a plurality of columnar parts 30 aligned in nanochannel 22. Since a biomolecule may be heated by heater 14 and may be denatured as described herein, the rate of detecting or identifying, or both detecting and identifying, a biomolecule at a molecular level may be increased. In addition, since columnar parts 30 may be provided, covering member 20 which may comprise PDMS may be prevented from being deflected and adhering to the bottom of nanochannel 22 when covering member 20 is overlapped on silicon oxide film 18.

In some embodiments as shown in FIG. 1, wherein device 10 may be utilized for thermally denaturing a biomolecule as heater 14 and temperature sensor 16 may be arranged in a width direction of nanochannel 22, parameters for temperature regulation using temperature sensor 16 may be minimized. Thus, complex temperature regulation may not necessary.

In some embodiments as shown in FIG. 3, wherein device 10 may be utilized for thermally denaturing a biomolecule as heater 14 and temperature sensor 16 may be arranged in a longitudinal direction of nanochannel 22, heater 14 may be fully disposed in a width direction of nanochannel 22. Thus, a biomolecule flowing through nanochannel 22 may be efficiently heated and denatured.

In some embodiments as described herein above, wherein device 10 may be utilized for thermally denaturing a biomolecule, due to the low heat capacity of heater 14 and the localization of heating, faster operation and lower power consumption may be effectuated in comparison with a conventional heater, and no heat dissipater may be necessary. In addition, since observed temperature information may be fed back to a heater regulator, localized temperature regulation may be possible. Device 10 which may be useful for thermally denaturing a biomolecule may be anticipated to be applicable in simple biomolecular testing using chips and in devices embedded in next-generation biomolecular sequencers.

In some embodiments, a method for producing a device for thermally denaturing a biomolecule may include: disposing a resistive heater 14 on a substrate 12 having low thermal conductivity; disposing a temperature sensor 16 in juxtaposition with heater 14 on substrate 12; applying a silicon oxide film 18 on substrate 12, heater 14, and temperature sensor 16; forming a nanochannel 22 in a region in silicon oxide film 18, wherein the region may overlap with heater 14, and wherein the region may also overlap with temperature sensor 16; and wherein the region may overlap a covering member 20 on silicon oxide film 18. In some embodiments, a method for producing a device 10 for thermally denaturing a biomolecule may comprise preparing a plurality of columnar parts 30 in nanochannel 22, wherein at least two columnar parts 30 may be aligned in a longitudinal direction and wherein at least two columnar parts 30 may be aligned in a width direction of nanochannel 22.

In some embodiments, heater 14 and temperature sensor 16 may be, for example, formed by electron beam lithography and physical vapor deposition (PVD), such as, e.g., sputtering. Silicon oxide film 18 may be, for example, formed by a vapor deposition technique, such as chemical vapor deposition (CVD), atomic layer deposition (ALD), or plasma-enhanced variants thereof. The heater 14 can be formed by PVD of metal elements comprising the heater 14. The temperature sensor 16 can be formed by PVD of metal elements comprising the temperature sensor 16. If the heater 14 or temperature sensor 16 comprises multiple metal elements, then multiple vapor sources may be used.

In some cases, vapor phase deposition is accompanied by annealing to an elevated temperature. For example, a metallic layer (e.g., for the heater 14 or temperature sensor 16) may be deposited by PVD at 250 K. The metallic layer may be subsequently annealed to a temperature of at least about 500 K or 600 K to anneal the layer. The layer may then be patterned (e.g., by photolithography) to define the feature being formed.

In some embodiments, nanochannel 22 and columnar parts 30 may be, for example, formed by treating silicon oxide film 18 with reactive ion etching after drawing a pattern by electron beam lithography.

In some embodiments utilizing a method for producing a device for thermally denaturing a biomolecule, it is possible to produce a device for thermally denaturing a biomolecule that can use a smaller sample volume for detection or identification, or for both detection and identification of a biomolecule, and may increase the rate of denaturing of a biomolecule. In addition, in some embodiments utilizing a method for producing a device for thermally denaturing a biomolecule, it is possible to produce a device for thermally denaturing a biomolecule that can increase a rate of detecting or identifying, or both detecting and identifying, a biomolecule at a molecular level.

The denaturing temperature of DNA is typically lower at low ionic concentrations. In addition, the persistence length is longer. In some embodiments it may be desirable to utilize single stranded DNA (ssDNA). In some embodiments it is desirable to have a longer persistence length to help maintain the linearity of ssDNA. In some embodiments, substantially low ionic concentration fluids such as deionized water or solutions of aqueous and non-aqueous fluids may be utilized. In some embodiments, low ionic concentration fluids may have total ionic concentrations less than or equal to about 10 mM, 1 mM, 100 μm, 50 μm, 10 μm, 5 μm, 1 μm, 0.5 μm, or 0.1 μm. A low ionic concentration fluid may have a total ionic concentration that is greater than or equal to about 0.001 μm, 0.01 μm, 0.1 μm, or 1 μM. In some cases, a low ionic concentration fluid has a total ionic concentration from about 0.001 μM to 10 mM, 0.01 μM to 1 mM, or 0.1 μM to 10 μm.

Lower temperatures may result in less Brownian motion or other molecular motions. These motions can cause an increase in measurement noise. It may be desirable to have a higher temperature at a region of a system wherein DNA may be denatured and a lower temperature at a region of a system wherein DNA or ssDNA may be measured. In some embodiments multiple temperature control zones may exist along a nanochannel. In some embodiments temperature control zones may be controlled to different temperatures. In some embodiments a temperature control mechanism external to the substrate may be used in addition to an embedded heater element. In some embodiments an external temperature control mechanism may provide subambient temperatures by removal of energy, utilizing, for example, a Peltier device.

Once denatured, ssDNA may be detected by tunneling current using nanoelectrode pairs. Current may be a function of temperature, so temperature control of the environment of a nanoelectrode pair may be desirable. In some embodiments one or more nanoelectrode pairs may exist in a channel, such as a nanochannel. In some embodiments, control of the temperature in a region associated with a nanoelectrode may be controlled by a local resistive heater.

In some cases, electrodes detect a current across a gap when a target species (e.g., a biomoleule, such as DNA or RNA) is disposed therebetween. The gap may be across a channel, such as a nanochannel. The current can be a tunneling current. Such a current can be detected upon the flow of the target species through the channel. In some cases, a sensing circuit coupled to the electrodes provides an applied voltage across the electrodes to generate a current. As an alternative or in addition to, the electrodes can be used to measure and/or identify the electric conductance associated with the target species (e.g., a base of a nucleic acid molecule). In such a case, the tunneling current can be related to the electric conductance.

In some embodiments, it may be desirable to utilize multiple methods to linearize DNA, for example, it may be desirable to utilize a set of linearization posts in combination with a region of elevated temperature, which may, for example, be effectuated by a heater in proximity to the set of linearization posts. A linearization post may be combined with a low ionic strength buffer. In some cases, a combination of linearization posts, temperature elevation in proximity to the set of linearization posts, and low ionic buffer.

In some embodiments it may be desirable to minimize the number of external connections required for a device. In some embodiments a resistive heater and temperature sensor may use the same resistive element by, for example, measuring the temperature of the resistive element when the heater is not active, for example, if off periods when a resistive element may have an applied voltage or current pulse width modulated. In some embodiments the resistive element may comprise platinum. In some embodiments, multiple heater elements may be linked together in, for example, in series to reduce the number of external connections.

In some embodiments heater(s) may be situated proximate to a channel between an input and a linearization feature(s). In some embodiments a heater may be situated proximate to a channel wherein a linearization feature resides. In some embodiments a heater may be situated proximate to both a linearization feature and a channel between an input and a linearization feature(s).

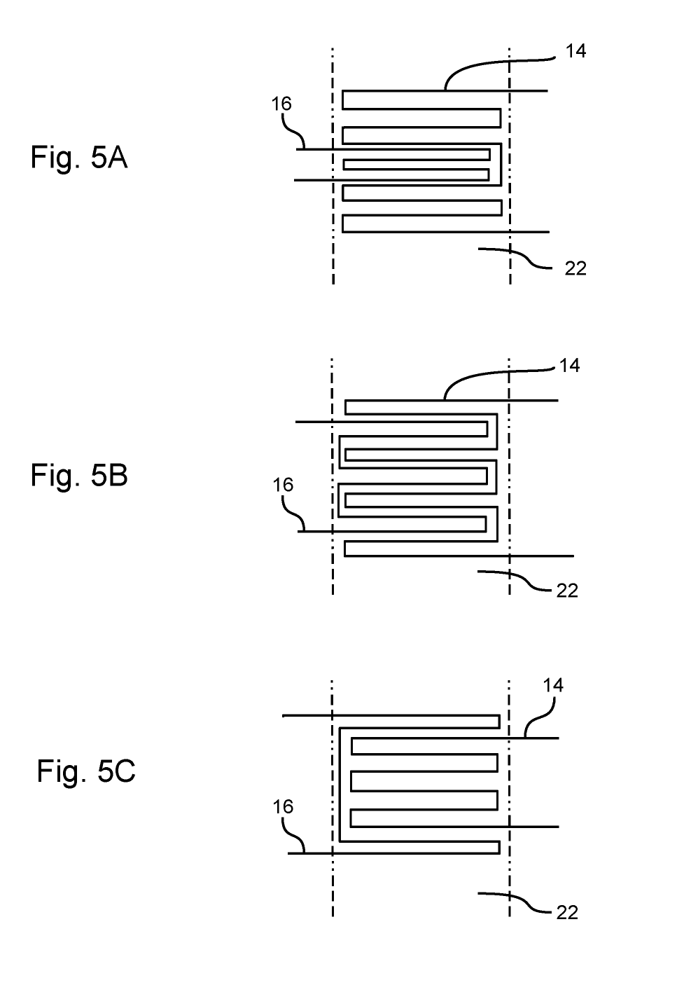

In some embodiments it may be desirable to linearize DNA before denaturation due to the much longer persistence length of double stranded DNA (dsDNA). In some embodiments the heater may be located downstream or between a linearization feature and a channel and associated electrode pair(s). In some embodiments it may be desirable to for a temperature sensor to be near the heater element, whereby improved temperature control may be effectuated. In some embodiments a temperature sensor may be located on, above, or below a heater. In some embodiments a temperature sensor 16 may be on the same plane as a heater 14, but may be internal to a heater zone as shown in FIG. 5A (columnar features deleted for clarity). In some embodiments a temperature sensor may be interdigitated with a heater as shown in FIG. 5B. In some cases, the heater 14 may overlap with a temperature sensor, and may be separated by a layer of silicon oxide (or other oxide). In some embodiments a heater may be of the same length as a temperature sensor, or may be of a shorter or longer length than an associated temperature sensor. In some embodiments a material for a sensor and an associated heater may be the same, or may be different. For example, a sensor may comprise platinum, tantalum and/or tungsten. In another example, a heater may comprise aluminum and/or tungsten. In some embodiment a temperature sensor 16 may be external to a heater 14 as shown in FIG. 5C.

### EXAMPLE

Denaturation of a DNA fragment comprising 18 base pairs is observed by the use of device 10 for thermally denaturing a biomolecule, as shown in FIG. 1 and FIG. 2. The dimensions of various parts of device 10 utilized for thermally denaturing a biomolecule are as follows.

Channel:

w=25 μm

d=500 nm

Heater and temperature sensor:

a=20 μm

Each respective line width in plan view is 1 μm.

Silicon oxide film:

t=400 nm

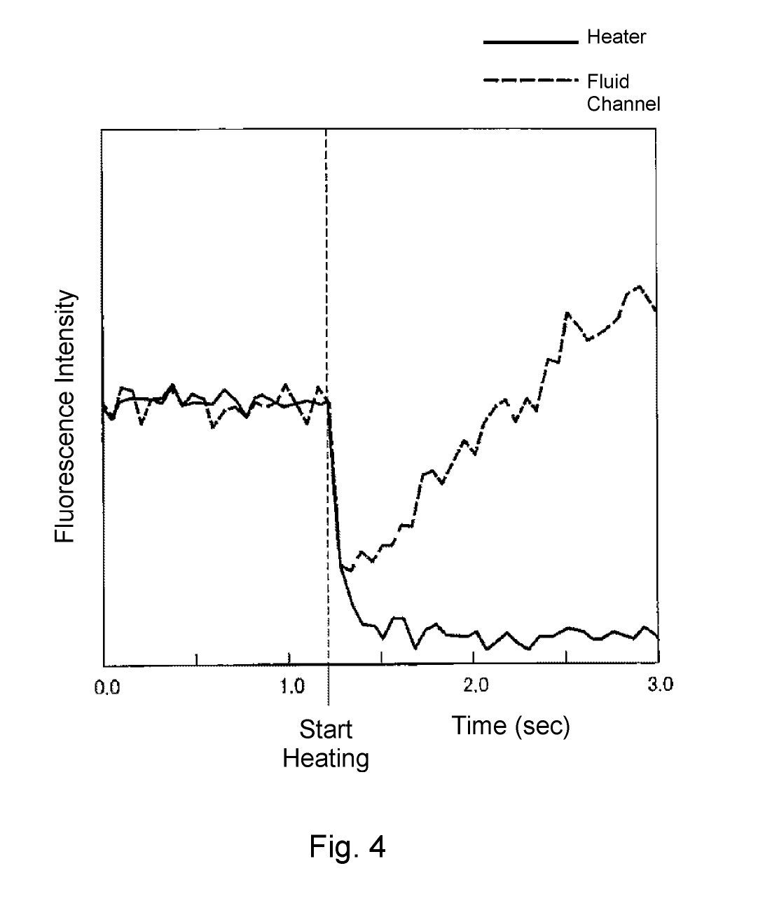

The DNA is synthesized with a fluorescent molecule and a quencher molecule at its terminals (e.g., quencher at 3′ end and fluorescent molecule at 5′ end, or quencher at 5′ end and fluorescent molecule at 3′ end), and is configured such that fluorescence is observed in a single-stranded state and is quenched in a double-stranded state. The DNA may be a double stranded DNA molecule or a hairpin. While a solution containing DNA fragments is caused to flow into channel 22 and is heated by heater 14, changes in fluorescent images are observed inside substrate 12 by the use of a total reflection fluorescence microscope. The result is shown in FIG. 4. In FIG. 4, the horizontal axis is time (in seconds) and the vertical axis is the amplitude of fluorescence. In addition, a solid line shows the amplitude of fluorescence of channel 22 at heater 14, and a dashed line shows the amplitude of fluorescence of channel 22 downstream from heater 14.

According to FIG. 4, since an increase of the amplitude of fluorescence in channel 22 is observed within 1 second from the start of heating by heater 14, it is demonstrated that DNA can be denatured rapidly. When heating is started, the amplitude of fluorescence of the background temporarily decreases due to the increase in temperature, and resultant changes in pH and concordant changes in fluorophore emission. However, since the amount of single-stranded DNAs in channel 22 increases over time, the amplitude of fluorescence increases. At heater 14, the amplitude of fluorescence does not increase because heater 14 reduces fluorescence emission as described herein.

Devices, systems and methods of the present disclosure may be combined with and/or modified by other devices, systems or methods, such as those described in U.S. Pat. No. 5,674,742, which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-75. (canceled)

76. A device for thermally denaturing a biomolecule, comprising:
a substrate having low thermal conductivity;
a resistive heater disposed adjacent to the substrate;
a temperature sensor disposed in juxtaposition with the resistive heater adjacent to the substrate;
a semiconductor oxide film adjacent to the resistive heater and the temperature sensor;
a nanochannel formed in at least a portion of the semiconductor oxide film; and
a covering member over at least a portion of the nanochannel.

77. The device of claim 76, wherein the nanochannel overlaps the resistive heater and the temperature sensor.

78. The device of claim 76, further comprising one or more columnar parts in the nanochannel.

79. The device of claim 78, wherein the one or more columnar parts comprise a plurality of columnar parts.

80. The device of claim 79, wherein at least two columnar parts of the plurality are aligned along a longitudinal direction of the nanochannel, and wherein at least two columnar parts of the plurality are aligned along a width direction of the nanochannel.

81. The device of claim 76, wherein the resistive heater and the temperature sensor are arranged along a width direction of the nanochannel.

82. The device of claim 76, wherein the resistive heater and the temperature sensor are arranged along a longitudinal direction of the nanochannel.

83. The device of claim 76, further comprising at least one pair of electrodes in fluid communication with the nanochannel, wherein the pair of electrodes detects a current across the nanochannel.

84. The device of claim 83, wherein the at least one pair of electrodes is in the nanochannel.

85. The device of claim 83, wherein the at least one pair of electrodes is separated by a gap having a distance that is less than or equal to about 2 nanometers.

86. The device of claim 83, wherein the at least one pair of electrodes is separated by a gap having a distance that is less than a diameter of the biomolecule.

87. The device of claim 76, further comprising a biomolecule that is suspended in a low ionic concentration fluid.

88. The device of claim 76, wherein the resistive heater is proximate to the nanochannel.

89. The device of claim 76, wherein the resistive heater overlaps the nanochannel.

90. The device of claim 76, wherein the resistive heater is adapted for use in heating and temperature sensing.

91. The device of claim 76, further comprising a plurality of resistive heaters that generate at least two temperature zones.

92. The device of claim 76, wherein the substrate has a thermal conductivity that is less than or equal to about 100 W/(mK).

93. A method for denaturing a biomolecule, comprising:
(a) providing a device having (i) a substrate having low thermal conductivity, (ii) a resistive heater disposed adjacent to the substrate, (iii) a temperature sensor disposed in juxtaposition with the resistive heater adjacent to the substrate, (iv) a semiconductor oxide film adjacent to the resistive heater and the temperature sensor, (v) a nanochannel formed in at least a portion of the semiconductor oxide film, and (vi) a covering member over at least a portion of the nanochannel;
(b) directing the biomolecule through the nanochannel; and
(c) using the resistive heater to apply heat to the biomolecule.

94. The method of claim 93, further comprising using at least one pair of electrodes to measure a current across a gap that separates the at least one pair of electrodes, wherein the current is a tunneling current.

95. A method for forming a device that thermally denatures a biomolecule, comprising:
(a) disposing a resistive heater adjacent to a substrate having low thermal conductivity;
(b) disposing a temperature sensor in juxtaposition with the resistive heater adjacent to the substrate;
(c) providing a semiconductor oxide film adjacent to the substrate, the resistive heater and the temperature sensor;
(d) forming a nanochannel in at least a portion of the semiconductor oxide film; and
(e) providing a covering member over at least a portion of the nanochannel.
